(19) United States
(12) Patent Application Publication
(10) Pub. No.: US 2003/0144269 A1
(43) Pub. Date: Jul. 31, 2003

(54) Reducing pulse pressures and vascular stiffness in hypertensive patients by administering a vasopeptidase inhibitor

(76) Inventor: Alan J. Block, Holland, PA (US)

Correspondence Address: STEPHEN B. DAVIS, BRISTOL-MYERS SQUIBB COMPANY, PATENT DEPARTMENT, P O BOX 4000, PRINCETON, NJ, 08543-4000, US

(21) Appl. No.: 10/322,275

(22) Filed: Dec. 18, 2002

Related U.S. Application Data
(60) Provisional application No. 60/342,924, filed on Dec. 20, 2001.

(51) Int. Cl. A61K 31/554

(52) U.S. Cl. 514/211.05

(57) ABSTRACT

Vasopeptidase inhibitors, especially omapatrilat, are useful in reducing central and peripheral pulse pressure and vascular stiffness in hypertensive patients. The vasopeptidase inhibitor may be used in combination with other pharmaceutically active agents.

This application claims priority from U.S. Serial No. 60/342,924 filed Dec. 20, 2001.

### BACKGROUND OF THE INVENTION

Over the last several years compounds have been reported in the patent and technical literature as possessing in a single molecule both angiotensin converting enzyme (ACE) inhibitory activity and neutral endopeptidase (EC24.11; NEP) inhibition activity. These compounds are of interest as cardiovascular agents particularly in the treatment of hypertension, congestive heart failure, and renal disease. These compounds are also referred to as vasopeptidase, dual metalloprotease, NEP/ACE, or ACE/NEP inhibitors.

Omapatrilat is such a vasopeptidase inhibitor which is currently undergoing clinical evaluation. Omapatrilat has the chemical name [4S-[4α(R*),7α,10aβ]]-octahydro-4-[(2-mercapto-1-oxo-3-phenylpropyl)amino]-5-oxo-7H-pyrido[2,1-b][1,3]thiazepine-7-carboxylic acid and the structural formula

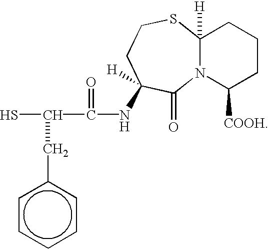

Omapatrilat, its preparation, and its use in treating cardiovascular diseases are disclosed by Robl in U.S. Pat. No. 5,508,272.

Gemopatrilat is another vasopeptidase inhibitor which is currently undergoing clinical evaluation. Gemopatrilat has the chemical name [S-(R*,R*)]-hexahydro-6-[(2-mercapto-1-oxo-3-phenylpropyl)amino]-2,2-dimethyl-7-oxo-1H-azepine-1-acetic acid and the structural formula

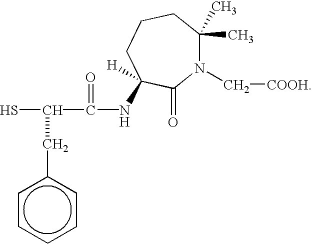

Gemopatrilat, its preparation, and its use in treating cardiovascular diseases are disclosed by Karanewsky et al. in U.S. Pat. No. 5,552,397.

The use of vasopeptidase inhibitors, especially omapatrilat, to treat isolated systolic hypertension is disclosed by Reeves et al. in WO01/74348 published Oct. 11, 2001.

Mitchell et al., Circulation, Supplement 1, Vol. 100, No. 18, 1-646, abstract 3407 (November 1999) report that in patients who had left ventricular dysfunction and class II-IV congestive heart failure treatment with omapatrilat produced a favorable trend toward reduction in carotid-femoral pulse wave velocity consistent with a pressure-independent reduction in aortic stiffness.

The use of vasopeptidase inhibitors, especially omapatrilat, to treat angina pectoris is disclosed by Powell et al. in U.S. Pat. No. 6,140,319.

### SUMMARY OF THE INVENTION

This invention is directed to the use of a vasopeptidase inhibitor to reduce central and peripheral pulse pressure and vascular stiffness in hypertensive patients. Preferred vasopeptidase inhibitors for this use are omapatrilat or a pharmaceutically acceptable salt thereof, gemopatrilat or a pharmaceutically acceptable salt thereof, or mixtures thereof. Most preferred is the use of omapatrilat.

The vasopeptidase inhibitor or inhibitors can also be employed in combination with other types of pharmaceutically active agents such as other types of antihypertensive agents and/or agents known to be useful in reducing the frequency or severity of stroke and/or coronary disease. The combination therapy can utilize a single dose form containing the vasopeptidase inhibitor or inhibitors or a pharmaceutically acceptable salt thereof, and the other pharmaceutically active agent or agents, co-administration of separate doses of each active agent, or administration of separate doses of each active agent according to a staggered schedule.

### DETAILED DESCRIPTION OF THE DRAWINGS

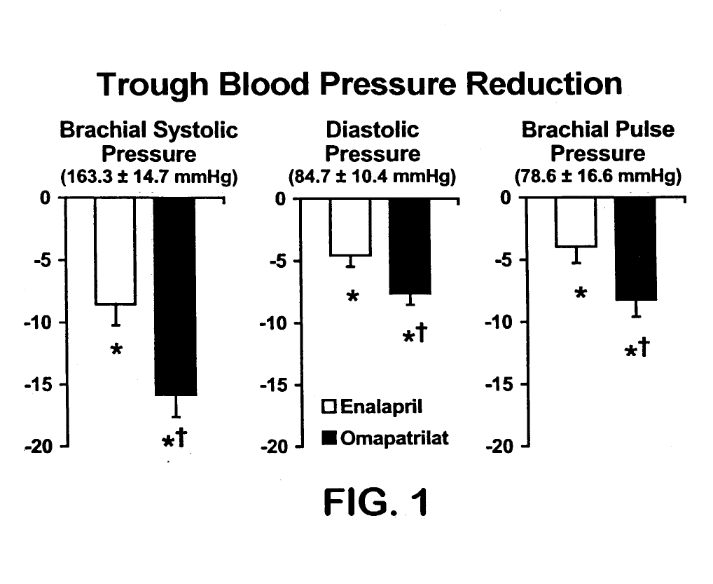

FIG. 1 shows change in peripheral (brachial) systolic, diastolic, and pulse pressure. Omapatrilat caused greater reductions in all measurements (p<0.05) than enalapril. (Numbers above each graphic are baseline values. Symbols refer to statistical analyses: asterisk, p<0.05 compared to baseline; dagger, p<0.05 compared to enalapril).

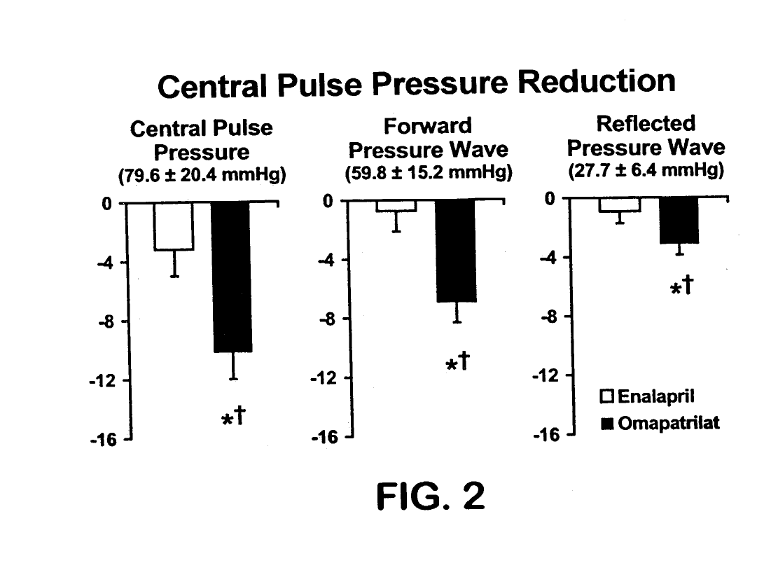

FIG. 2 shows changes in central pulse pressure, forward pressure wave, and reflected pressure wave. As central pulse pressure is influenced by both the forward and reflected wave, the greater reduction in the central pulse pressure compared to enalapril is explained predominantly by the significant reductions by omapatrilat on the forward and reflected pressure waves.

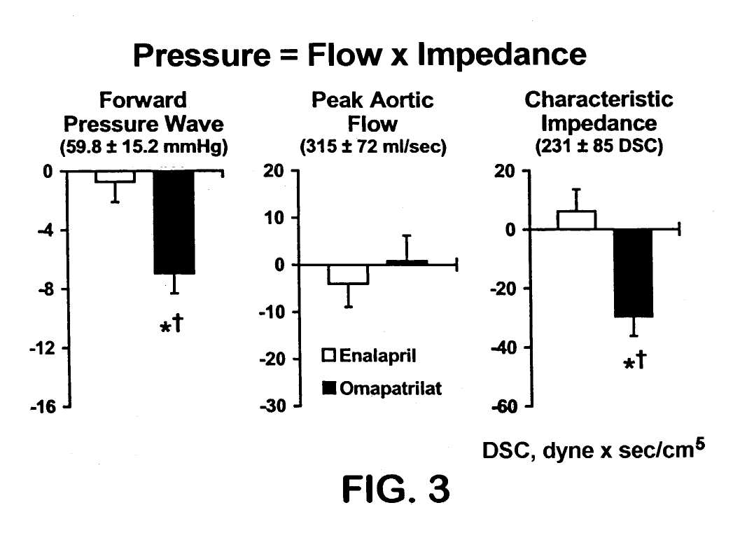

FIG. 3 shows that characteristic impedance, a direct measure of aortic stiffness, is significantly reduced by omapatrilat without a change in aortic flow.

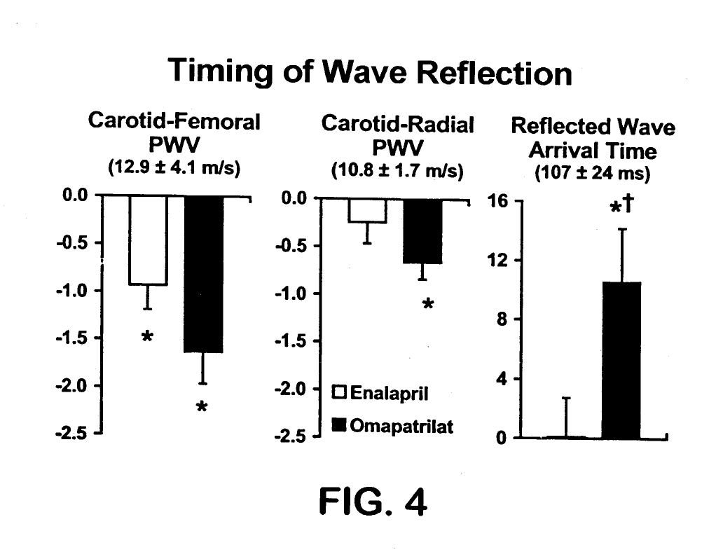

FIG. 4 shows changes in regional pulse wave velocities (carotid-femoral and carotid-radial) and arrival time of the reflected wave. While both omapatrilat and enalapril reduced regional pulse wave velocity, only omapatrilat delayed reflected wave arrival time (p<0.05) due to a significant reduction in vascular stiffness (i.e., characteristic impedance—FIG. 3).

### DETAILED DESCRIPTION OF THE INVENTION

Pulse pressure has emerged as a strong independent predictor of cardiovascular events in patients with hypertension. Elevated pulse pressure is presumed to be an indicator of increased vascular stiffness, which increases the amplitude of the pressure pulse produced by a given flow wave in the central aorta. Furthermore, the propagation velocity of pressure waves is faster in stiff vessels, leading to earlier return of reflected pressure waves to the central aorta, further augmenting central systolic and pulse pressure and left ventricular load. Such increased pulsatile load could promote ventricular and vascular hypertrophy and fibrosis, endothelial dysfunction, and atherogenesis, and may explain the observed association between higher pulse pressure and increased clinical events.

This invention is directed to the use of one or more vasopeptidase inhibitors to treat hypertensive patients to cause a pressure-independent reduction in vascular stiffness and result in a reduction in pulse pressure. Angiotension converting enzyme (ACE) inhibitors are thought to reduce the stiffness of central conduit vessels, possibly by opposing the vasoconstrictive, hypertrophic and profibrotic effects of angiotensin II on the conduit vessell wall and endothelium, see Safar et al., Journal of Vascular Research, 34, p. 67-81 (1997). Vasopeptidase inhibitors are a single molecule that inhibits both angiotensin converting enzyme and neutral endopeptidase, an enzyme that inactivates several vasodilatory peptides. As a result of these combined actions, vasopeptidase inhibitors reduce levels of angiotensin II and increase levels of vasodilatory peptides including atrial, brain and C-type natriuretic peptides, bradykinin and adrenomedullin and have an enhanced effect on central and peripheral pulse pressure and a favorable pressure-independent change in central aortic stiffness.

Preferred vasopeptidase inhibitors for use in reducing pulse pressure and vascular stiffness according to this invention are omapatrilat or a pharmaceutically acceptable salt thereof and gemopatrilat or a pharmaceutically acceptable salt thereof, particularly omapatrilat. The vasopeptidase inhibitor can be administered to a hypertensive patient in an amount ranging from about 2.5 mg to about 240 mg per 24 hours, preferably from about 10 to about 120 mg per 24 hours. The vasopeptidase inhibitor can be administered in one or more doses over the 24 hour period to provide the total amount of active agent within the above range. If more than one dose is administered per 24 hours, the doses may be equal or may be varied in strength. Of course, the amount of active agent employed will be adjusted by the physician and may initially be at the low end of the above range and gradually increased. Also, if a combination of vasopeptidase inhibitors is employed, then one or both of the inhibitors may be administered in a lesser amount provided that the total combination of active agents administered is within the above range.

The vasopeptidase inhibitor is preferably administered orally in tablet or capsule form. However, other methods of administration may also be utilized including sublingually, bucally, parenterally such as by subcutaneous, intraveneous, or intramuscular injection or infusion techniques, nasally such as by inhalation spray, topically such as in the form of a cream or ointment, transdermally as in the form of a patch that is applied to the skin, or rectally such as in the form of suppositories. The various dosage formulations contain in addition to the vasopeptidase inhibitor conventional pharmaceutically acceptable vehicles, stabilizers, preservatives, lubricants, diluents, and other conventional ingredients. The formulation may be administered for immediate release or extended release.

Another aspect of this invention is the reduction of pulse pressure and vascular stiffness with one or more vasopeptidase inhibitors, as described above, in combination with other types of pharmaceutically active agents. For example, other antihypertensive agents can be utilized in combination with the vasopeptidase inhibitors. Suitable agents include diuretics such as hydrochlorothiazide, which is preferred, and bendroflumethiazide, α- and/or β-adrenergic blocking agents such as propranolol hydrochloride, timolol maleate, carvedilol, metoprolol tartrate and atenolol, calcium entry blockers such as amlodipine besylate, diltiazem hydrochloride, and verapamil hydrochloride, and angiotensin II receptor antagonists such as irbesartan, losartan, valsartan, candesartan cilexetil, and eprosartan. Agents known to be useful in reducing the frequency or severity of stroke and/or coronary disease can also be utilized in combination with the vasopeptidase inhibitors. Suitable agents include cholesterol reducing agents particularly HMG-CoA reductase inhibitors such as pravastatin sodium, simvastatin, lovastatin, atorvastatin calcium, and fluvastatin sodium, and platelet aggregation inhibitors such as clopidogrel bisulfate, ticlopidine hydrochloride and aspirin.

In such combination therapies, the amount of other pharmaceutically active agents employed is that previously approved for the treatment of hypertension or the reduction of the frequency or severity of stroke and/or coronary disease. Lesser amounts of the other pharmaceutically active agent may be employed as determined by the treating physician. Also, in the combination therapy, the amount of vasopeptidase inhibitor may be less than the amount employed in the monotherapy described above.

The vasopeptidase inhibitor and the other pharmaceutically active agent or agents may be formulated as a single dosage form, may be co-administered from separate dosage forms, or may be administered from separate dosage forms according to a staggered schedule.

The term pharmaceutically acceptable salt includes alkali metal salts such as sodium and potassium, alkaline earth metal salts such as calcium and magnesium, salts derived from amino acids such as arginine, lysine, etc. and salts derived from amines such as alkylamines, e.g. t-butylamine, t-amylamine, etc., substituted alkylamines, e.g. benzylamine, dialkylamines, substituted dialkylamines, e.g. N-methyl glucamine, trialkylamines, substituted trialkylamines, and quaternary ammonium salts.

### EXAMPLE

The following study was performed to compare treatment with the vasopeptidase inhibitor omapatrilat and the angiotensin converting enzyme inhibitor enalapril.

Study subjects. Male or female subjects age 18 years or older were eligible if they were in sinus rhythm and had systolic or mixed systolic-diastolic hypertension, defined as a seated systolic blood pressure ≧160 mmHg if newly diagnosed or ≧140 mmHg if currently treated and diastolic blood pressure ≦110 mmHG. Patients with known or suspected secondary forms of hypertension were excluded. Patients with a history of transient ischemic attacks within the prior 12 months or myocardial infarction or angina within the prior 6 months were excluded. All patients with heart failure, documented ejection fraction <45%, valvular heart disease or clinically significant peripheral vascular disease (bruit or diminished pulse in any of the brachial, radial, femoral or carotid arteries or blood pressure difference between upper extremities >6 mmHg) were excluded. Additional exclusion criteria included renal or renovascular disease, uncontrolled diabetes (glucose >200 mg/dl fasting or >220 mg/dL nonfasting), autoimmune disease, multiple drug allergies, bronchospastic disease requiring chronic medication. Laboratory exclusion criteria included hemoglobin <9 g/dl, platelet count <80,000/μl, white cell count <3000/μl, neutrophil count <1500/μl, serum potassium <3.5 or >5.2 mmol/L, alanine aminotransferase or aspartate aminotransferase greater than three times the upper limits of normal or serum creatinine >2.5 mg/dL. Failure to obtain a technically satisfactory baseline study, as determined by the core lab, was an exclusion criterion. If an initial baseline study had potentially remediable technical limitations, a repeat study was allowed within 2-9 days.

Treatment protocol: A single-blind placebo lead-in period of 1-2 weeks, during which the seated blood pressure was confirmed to be ≧160 mmHg and <200 mmHg and the diastolic pressure ≦110 mmHg, after withdrawl of antihypertensive medications, was followed by a double-blind active treatment period of 12 weeks. Baseline hemodynamic studies were performed at the end of the placebo lead-in period between 6:00 and 10:00 a.m. with the patient in a fasting state prior to taking morning medications and off all antihypertensive medications for at least 1 week. Patients were randomized within 2-9 days following an approved baseline study. Treatment was initiated with either 10 mg omapatrilat or 10 mg enalapril and was titrated at two and four weeks to doses of 40 then 80 mg omapatrilat or 20 then 40 mg enalapril. Patients who failed to tolerate at least 40 mg omapatrilat or 20 mg enalapril were excluded from the study. No concomitant antihypertensive medications were permitted during the study. A trough hemodynamic study, the primary endpoint of the trial was performed at the end of the active treatment period, 24 hours after the last prior dose of study medication, under identical conditions as the baseline study. Patients were then given one additional dose of study medication, which was followed 4 hours later by a final (peak) hemodynamic study. In order to focus on long-term effects of therapy, the primary endpoint of the study was prospectively defined as the trough change from baseline in pulsatile hemodynamics.

Hemodynamic data acquisition: Subjects were studied in the supine position after approximately 10 minutes of rest. Using a semi-automated computer-controlled device, auscultatory blood pressure was obtained 3-5 times at 2 minute intervals with a goal of obtaining 3 sequential readings that agreed to within 5 mmHg for systolic and diastolic. Arterial tonometry and ECG were obtained form the brachial, radial, femoral and carotid arteries using a custom transducer. Next, the patient was placed in the left lateral decubitus position and echocardiographic images of the left ventricular outflow tract were obtained from a parasternal long axis view. This was followed by duplicate acquisitions of simultaneous tonometry of the carotid artery and pulsed Doppler of the left ventricular outflow tract from an apical 5-chamber view. Finally, body surface measurements from suprasternal notch to radial, femoral and carotid recording sites were obtained. All data were digitized during the primary acquisition (ECG and tonometry pressures at 1000 Hz, audio at 12 kHz, and video at 30 frames/s), transferred to CD-ROM and shipped to the core lab for analysis.

Data analysis: All studies were analyzed at the core lab in a blinded fashion and all decisions on technical adequacy of studies were made prior to unblinding. Tonometry waveforms were signal-averaged using the ECG as a fiducial point, Mitchell et al., Am. J. Physiol. 267, H1907-H1915 (1994). Blood pressures were over-read by two reviewers at the core lab. Average systolic and diastolic cuff pressures were used to calibrate the peak and trough of the signal-averaged brachial pressure waveform. Diastolic and integrated mean brachial pressures were then used to calibrate carotid, radial and femoral pressure tracings, Kelly et al, J. Am. Coll. Cardial., 20, 952-963 (1992). Pulse wave velocities from carotid to radial and femoral sites were calculated from the delay between the appearance of the pressure waveform foot in the carotid and periphery, Mitchell et al, J. Appl. Physiol, 82, 203-210 (1997). The distance between recording sites was adjusted for parallel transmission in the aorta and carotid by subtracting the suprasternal notch-to-carotid distance from the suprasternal notch-to-peripheral site distances, Mitchell et al., “Pulsatile Hemodynamics In Congestive Heart Failure”, Hypertension, 2001 (In press). These corrected distances were divided by respective foot-to-foot transmission delays to give pulse wave velocity. The time delay from the foot of the carotid waveform to the inflection point between forward and reflected pressure waves was identified and used as a measure of the timing of wave reflection. Systolic ejection period was measured from the foot of the waveform to the dicrotic notch. Augmentation index was calculated as previously described, Murgo et al., Circulation, 62, 105-116 (1980).

Left ventricular volume flow was calculated by spectral analysis of the digitized broadband Doppler audio signal as previously described, Mitchell et al., Hypertension, 2001 (supra). Characteristic impedance was estimated in the time domain as previously described, Mitchell et al., Hypertension, 2001 and Lucas et al, IEEE Trans BME, 35, 62-68 (1988). Briefly, the rise time from the onset of flow to the point where flow achieved 95% of its peak value was determined. The corresponding pressure rise during this interval was determined and characteristic impedance was calculated by dividing the change in pressure by the corresponding change in flow. The early phase of the ejection period must be used to differentiate the primary pressure rise, which is the product of characteristic impedance and flow, from the secondary pressure rise, which is due to a combination of ongoing forward flow and reflected pressure wave. Pressure waveforms were decomposed into forward and backward (reflected) waves in the time domain, Westerhof et al., Cardiovasc. Res., 6, 648-656 (1972); the ratio of their amplitudes was taken as an index of global reflection and the extent of their temporal overlap, expressed as a percentage of systolic ejection period, was taken as an index of abnormal reflected wave timing. Proximal aortic compliance per unit length, C₁ was calculated as previously described, Mitchell et al, Circulation, 94, 2923-2929 (1996).

The quality of tonometry waveforms was assessed by evaluating the foot of the waveform, initial upstroke, dicrotic notch and diastolic contour. Improper centering of the pulse transducer or inadequate hold-down pressure produces a low amplitude waveform with a poorly defined foot and dicrotic notch. Excessive hold-down pressure can distort the waveform foot and upstroke and can create a premature minimum in mid-diastole. Flow waveforms were evaluated for beat-beat consistency of flow peaks, absence of mitral inflow signal and evidence of proper positioning of the sample volume in the left ventricular outflow tract on accompanying video images.

Statistical analysis: Baseline characteristics were tabulated and compared using a chi-square statistic for dichotomous variables and analysis of variance for continuous variables. Significance levels of between group comparisons of the change from baseline in continuous variables were assessed by a general linear model that adjusted for baseline value. Pressue-independence of changes in stiffness parameters was assessed by including baseline mean pressure and change in mean pressure in the model. Values are presented as the mean±the standard deviation except as noted. A two-sided P<0.05 was considered significant.

Results

Of 335 enrolled consenting patients, a total of 213 completed the single-blind lead-in period, met all entry criteria and were randomized to active treatment. Of these, 185 subjects completed the active treatment period and 167 had a technically adequate paired assessment of all baseline and follow-up hemodynamic parameters. Baseline characteristics of the two treatment cohorts were similar and consistent with a population having moderate systolic hypertension and elevated peripheral (brachial) pulse pressure (Tables 1-3). There were no differences between treatment groups in any baseline hemodynamic parameters (Tables 2-3).

As anticipated, both therapies reduced systolic, diastolic and mean arterial pressure. However, treatment with omapatrilat for 12 weeks resulted in greater reductions in all trough blood pressure parameters, including peripheral and central pulse pressure (Table 2). Neither treatment had an effect on heart rate or peak aortic flow, whereas peripheral resistance was reduced more by omapatrilat. Omapatrilat markedly reduced the amplitude of the forward pressure wave by reducing aortic stiffness (characteristic impedance) in the setting of unchanged peak flow (Tables 2-3, FIG. 3). There were trends toward greater reductions in carotid-radial and carotid-femoral pulse wave velocity with omapatrilat, suggesting an enhanced effect on the more distal aorta and peripheral conduits as well (FIG. 4).

The agents comparably reduced augmentation index (Table 3). Reduced augmentation with omapatrilat was associated with increased reflected wave transit time, indicating later return of the reflected wave (FIG. 4), and reduced temporal overlap between forward and reflected wave (Table 3). In contrast, reduced augmentation with enalapril was associated with shortening of the systolic ejection period, which was not seen with omapatrilat (Table 3).

Since many measures of conduit vessel stiffness are pressure-dependent, the change in characteristic impedance was further adjusted for baseline mean arterial pressure and change in mean arterial pressure. The favorable change in characteristic impedance with omapatrilat remained significant (P<0.05). Furthermore, when only those subjects with a reduction in mean arterial pressure between 0 and −40 mmHg were evaluated (68 assigned to omapatrilat and 59 assigned to enalapril), the reduction in mean arterial pressure did not differ (−13.8±7.4 vs. −12.0±8.1 mmHg, P=0.112). However, the differential change in characteristic impedance remained highly significant (−37±58 dyne×sec/cm⁵ with omapatrilat vs. −4±67 dyne×sec/cm⁵ with enalapril, P=0.005). These analyses demonstrate that 12 weeks of vasopeptidase inhibition, as compared to converting enzyme inhibition alone, had a differential effect on the pressure-stiffness relationship of the proximal aorta, resulting in a greater reduction in stiffness for a given change in mean pressure with omapatrilat.

TABLE 1
Baseline characteristics

| Variable | Enalapril | Omapatrilat | P |
|---|---|---|---|
| N | 87 | 80 | — |
| Age (years) | 61 ± 10 | 61 ± 9 | 0.801 |
| Height (cm) | 169 ± 10 | 169 ± 9 | 0.773 |
| Weight (kg) | 87 ± 18 | 83 ± 17 | 0.132 |
| Body mass index (kg/m²) | 30 ± 5 | 29 ± 6 | 0.206 |
| Male (%) | 64 | 61 | 0.677 |
| History of (%): | | | |
| Coronary disease | 3 | 5 | 0.617 |
| Diabetes | 5 | 13 | 0.066 |
| Active smokers | 12 | 13 | 0.841 |
| Concomitant medications (%): | | | |
| Lipid lowering agent | 26 | 24 | 0.689 |
| Aspirin | 21 | 23 | 0.776 |
| Estrogen (% of women) | 39 | 39 | 1.000 |

TABLE 2
Hemodynamic parameters.

| | Enalapril | | Omapatrilat | | |
|---|---|---|---|---|---|
| | Baseline | 12 weeks | Baseline | 12 weeks | P^{‡} |
| Brachial pressures (mmHg) | | | | | |
| Systolic pressure | 163 ± 15 | 155 ± 20^{†} | 163 ± 15 | 147 ± 20^{†} | 0.003 |
| Diastolic pressure | 85 ± 10 | 81 ± 10^{†} | 84 ± 11 | 76 ± 11^{†} | 0.004 |
| Pulse pressure | 78 ± 16 | 74 ± 20^{†} | 79 ± 17 | 71 ± 18^{†} | 0.028 |
| Mean pressure | 117 ± 10 | 111 ± 12^{†} | 117 ± 10 | 105 ± 13^{†} | 0.001 |
| Central hemodynamics | | | | | |
| Systolic pressure (mmHg) | 164 ± 18 | 156 ± 24^{†} | 165 ± 19 | 147 ± 24^{†} | 0.001 |
| Pulse pressure (mmHg) | 79 ± 19 | 75 ± 23 | 81 ± 22 | 71 ± 22^{†} | 0.010 |
| Heart rate (min⁻¹) | 63 ± 8 | 63 ± 8 | 64 ± 9 | 64 ± 9 | 0.879 |
| Mean flow(ml/s) | 76 ± 17 | 73 ± 16^{†} | 75 ± 19 | 73 ± 16 | 0.530 |
| Peak flow(ml/s) | 318 ± 73 | 314 ± 70 | 311 ± 71 | 312 ± 68 | 0.655 |
| Peripheral resistance (DSC*) | 2172 ± 508 | 2122 ± 462 | 2208 ± 567 | 1986 ± 449^{†} | 0.004 |
| Central stiffness parameters | | | | | |
| First impedance modulus (DSC) | 354 ± 139 | 352 ± 163 | 374 ± 149 | 323 ± 119 | 0.004 |
| Characteristic impedance (DSC) | 225 ± 87 | 231 ± 94 | 237 ± 83 | 208 ± 70^{†} | 0.001 |
| Proximal aortic compliance (CD*) | 0.45 ± 0.24 | 0.49 ± 0.28 | 0.40 ± 0.19 | 0.53 ± 0.26^{†} | 0.021 |

*DSC, dyne × sec / cm⁵; CD, 10⁻⁵ cm⁴ / dyne
^{†}P < 0.05 for change from baseline within group
^{‡}P for difference between therapies in change from baseline, adjusted for baseline value

TABLE 3
Pulse wave velocity and waveform morphology

| | Enalapril | | Omapatrilat | | |
|---|---|---|---|---|---|
| | Baseline | 12 Weeks | Baseline | 12 Weeks | P^{‡} |
| Carotid-radial PWV (m/s) | 10.8 ± 1.8 | 10.6 ± 1.8^{†} | 10.8 ± 1.7 | 10.1 ± 1.4^{†} | 0.062 |
| Carotid-femoral PWV (m/s) | 12.8 ± 4.0 | 11.9 ± 3.7^{†} | 13.0 ± 4.1 | 11.4 ± 3.1^{†} | 0.076 |
| Reflected wave arrival time, tᵢ (ms) | 108 ± 26 | 108 ± 25 | 105 ± 23 | 116 ± 36^{†} | 0.024 |
| Systolic ejection period (ms) | 325 ± 25 | 319 ± 27^{†} | 323 ± 26 | 320 ± 26 | 0.193 |
| Pressure at tᵢ (mmHg) | 144 ± 14 | 139 ± 17^{†} | 145 ± 14 | 132 ± 20^{†} | 0.002 |
| End-systolic pressure (mmHg) | 127 ± 11 | 120 ± 14^{†} | 127 ± 13 | 114 ± 16^{†} | 0.001 |
| Augmentation index (%) | 25 ± 11 | 21 ± 10^{†} | 24 ± 10 | 19 ± 13^{†} | 0.256 |
| Forward wave, P_{f} (mmHg) | 59 ± 14 | 58 ± 17 | 61 ± 16 | 54 ± 16^{†} | 0.002 |
| Reflected wave, P_{b} (mmHg) | 27 ± 6 | 27 ± 8 | 28 ± 7 | 25 ± 7^{†} | 0.046 |
| P_{b}P_{f} Amplitude ratio (%) | 48 ± 8 | 46 ± 8 | 47 ± 7 | 47 ± 7 | 0.395 |
| P_{f}P_{b} Temporal overlap (%) | 67 ± 9 | 66 ± 8 | 67 ± 8 | 64 ± 12^{†} | 0.044 |

^{†}P < 0.05 for change from baseline within group
^{‡}P for difference between therapies in change from baseline, adjusted for baseline value

### DISCUSSION

The study compared the effects of vasopeptidase inhibition with omapatrilat and ACE inhibition with enalapril and demonstrated an enhanced effect of vasopeptidase inhibition on central and peripheral pulse pressure. The mechanism of this enhanced effect on pulse pressure was a pressure-independent reduction in characteristic impedance, a direct measure of proximal aortic stiffness, resutling in a smaller forward pressure wave for a given central aortic flow wave. This change in central aortic stiffness was accompanied by favorable changes in other measures of pulsatile load, including the first modulus of impedance, proximal aortic compliance, carotid-radial and carotid-femoral pulse wave velocities and timing of wave reflection. Importantly, the beneficial effects of omapatrilat on conduit vessel function were in addition to those achieved by ACE inhibition alone, suggesting an important role for the neutral endopeptidase-inhibiting effects of the vasopeptidase inhibitor omapatrilat in mediating the observed differential changes in pulsatile load. This study demonstrated a clear, pressure-independent pharmacologic reduction in proximal aortic stiffness in a hypertensive cohort by the vasopeptidase inhibitor omapatrilat.

What is claimed is:

1. A method of reducing central and peripheral pulse pressure and vascular stiffness in a hypertensive patient comprising administering an effective amount of vasopeptidase inhibitor.

2. The method of claim 1 wherein said vasopeptidase inhibitor is omapatrilat, a pharmaceutically acceptable salt of omapatrilat, gemopatrilat, a pharmaceutically acceptable salt of gemopatrilat, or mixtures thereof.

3. The method of claim 2 wherein said vasopeptidase inhibitor is omapatrilat.

4. The method of reducing central and peripheral pulse pressure and vascular stiffness in a hypertensive patient comprising administering an effective amount of a vasopeptidase inhibitor and another pharmaceutically active agent.

5. The method of claim 4 wherein said vasopeptidase inhibitor is omapatrilat, a pharmaceutically acceptable salt of omapatrilat, gemopatrilat, a pharmaceutically acceptable salt of gemopatrilat, or mixtures thereof.

6. The method of claim 5 wherein said vasopeptidase inhibitor is omapatrilat.

7. The method of claim 4 wherein said other pharmaceutically active agent is co-administered with said vasopeptidase inhibitor.

8. The method of claim 4 wherein said other pharmaceutically active agent is administered separately from said vasopeptidase inhibitor.

9. The method of claim 4 wherein said other pharmaceutically active agent is an antihypertensive agent and/or an agent known to be useful in reducing the frequency or severity of stroke and/or coronary disease.

10. The method of claim 9 wherein said antihypertensive agent is selected from the group consisting of diuretics, α- and/or β-adrenergic blocking agents, calcium entry blockers, angiotensin II receptor antagonists, and said agent known to be useful in reducing the frequency or severity of stroke and/or coronary disease is selected from the group consisting of HMG-CoA reductase inhibitors and platelet aggregation inhibitors.

11. The method of claim 10 wherein said diuretic is hydrochlorothiazide, said α- or β-adrenergic agent is selected from the group consisting of propanolol hydrochloride, timolol maleate, metoprolol tartrate, carvedilol, and atenolol, said calcium entry blocker is selected from the group consisting of amlodipine besylate, diltiazem hydrochloride, and verapamil hydrochloride, and said angiotensin II receptor antagonist is selected from the group consisting of irbesartan, losartan, valsartan, candesartan cilexetil, and eprosartan.

12. The method of claim 10 wherein said HMG-CoA reductase inhibitor is selected from the group consisting of pravastatin sodium, simvastatin, lovastatin, atorvastatin calcium, and fluvastatin sodium, and said platelet aggregation inhibitor is selected from the group consisting of clopidogrel bisulfate, ticlopidine hydrochloride and aspirin.

13. The method of claim 6 where said other pharmaceutically active agent is hydrochlorothiazide.
